(19) United States
(12) Patent Application Publication
(10) Pub. No.: US 2023/0321033 A1
(43) Pub. Date: Oct. 12, 2023

(54) THIOPHEN COMPOUNDS FOR USE IN THE TREATMENT OF RENAL FIBROSIS

(71) Applicant: ENYO PHARMA, LYON (FR)

(72) Inventors: RAPHAËL DARTEIL, LYON (FR); STÉPHANIE LE CORRE, LYON (FR); ERIC MELDRUM, RIEHEN (CH); FABIOLA TERZI, PARIS (FR); JACKY VONDERSCHER, SENOUILLAC (FR)

(21) Appl. No.: 18/019,486

(22) PCT Filed: Aug. 5, 2021
(86) PCT No.: PCT/EP2021/071829

§ 371 Date: Feb. 3, 2023

(30) Foreign Application Priority Data
Aug. 5, 2020 (EP) 20305901.9

(51) Int. Cl. A61K 31/381; A61K 31/5377; A61K 31/5386; A61P 13/12

(52) U.S. Cl. CPC A61K 31/381; A61K 31/5377; A61K 31/5386; A61P 13/12

(57) ABSTRACT

The present invention relates to a method for treating renal fibrosis.

### FIELD OF THE INVENTION

The present invention relates to the field of the medicine, in particular of chronic kidney disease.

### BACKGROUND OF THE INVENTION

Chronic kidney disease (CKD) is a long-term condition in which the kidneys do not work correctly. It has a high prevalence and estimated to affects hundreds of millions of people. Diabetes and hypertension are the two principal causes of CKD. Although the kidney has a capacity for regeneration after acute injury, in the context of chronic injury, regeneration and recovery are much more difficult. Accordingly, this process is often irreversible, leading to end-stage renal disease, a situation that requires dialysis or renal transplantation.

The progression of CKD is characterized by the loss of nephrons, the functional units of kidney, and their replacement by extracellular matrix (ECM), independently of the associated disease. Thus, one of the consequences of CKD is glomerulosclerosis and tubulointerstitial fibrosis caused by an imbalance between excessive synthesis and reduced breakdown of the ECM. There are currently no effective treatments for preventing the progression of renal fibrosis. The presence of fibrosis in CKD is strongly related to the future manifestation of renal failure and has thus been related with poor long-term prognosis.

More precisely, progressive CKD may be viewed as having three phases. First, there is cause-specific injury and acute response to that injury. In the second phase, misdirected repair generates fibrosis and dysfunction. At this phase, although fibrosis is a pathological and destructive event, it is essentially a self-limiting repair process to restrict the injury. The third and final stage is that of relatively steady progressive loss of remnant nephrons, which requires multiple nascent injury to each nephron or cluster of nephrons.

Current therapies have limited effectiveness and only delay disease progression, underscoring the need to develop novel therapeutic approaches to either stop or reverse progression. Therefore, it is a strong unmet need of treatment in order to achieve a reversion/elimination of renal fibrosis.

### SUMMARY OF THE INVENTION

The present application provides compounds capable of eliminating or reversing renal fibrosis. Up today, according to our knowledge, it is the first time that such an activity is reported. In addition to the effect on renal fibrosis, the compounds are further capable of significantly decreasing renal inflammation. The combined effect on the elimination of the renal fibrosis and the decreased renal inflammation is a significant advantage for improving renal function.

Accordingly, the present invention relates to
a compound of formula (I) for use for treating renal fibrosis in a subject, or
the use of a compound of formula (I) for use for treating renal fibrosis in a subject, or
a pharmaceutical composition comprising a compound of formula (I) for use for treating renal fibrosis in a subject,
a method for treating renal fibrosis in a subject, comprising administering a therapeutic amount of a compound of formula (I) to the subject, thereby decreasing or reversing renal fibrosis in the subject,

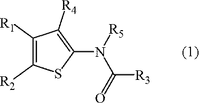

wherein:
R₁ represents a fused arylcycloalkyl or a cycloalkyl optionally substituted by at least one radical selected in the group consisting of:
   a halogen,
   a (C₁-C₆)alkyl or a (C₁-C₆)alkyloxy optionally substituted by at least one halogen, preferably optionally substituted by at least one fluorine, and
   a hydroxy,
   a -CO-R₆ or a -CO₂R₆ with R₆ being a hydrogen or a (C₁-C₆)alkyl, and
   an optionally substituted aryl;
R₂ represents:
   a hydrogen,
   a halogen,
   a (C₁-C₆)alkyl optionally substituted by at least one halogen, preferably optionally substituted by at least one fluorine,
   an optionally substituted aryl, or
   an optionally substituted cycloalkyl;
R₃ represents:
   a 5-10 membered ring, saturated or unsaturated selected in the group consisting of:
      an aryl optionally fused to a heterocycloalkyl, preferably selected from the group consisting of a dioxole, a morpholine, a dioxane, a tetrahydropyran, and a tetrahydrofuran,
      a heteroaryl,
      a cycloalkyl,
      a heterocycloalkyl, and
      a 5-10 membered bridged carbocyclyl or heterocyclyl, said 5-10 membered ring is optionally substituted by at least one radical selected in the group consisting of:
         a halogen,
         a (C₁-C₆)alkyl optionally substituted by at least one halogen, preferably optionally substituted by at least one fluorine, or by an optionally bridged heterocycloalkyl optionally substituted by a (C₁-C₆)alkyl,
         a -NH-(C₁-C₆)alkyl or a -N-((C₁-C₆)alkyl)₂, optionally substituted by at least one radical selected in the group consisting of a heterocycloalkyl, a cycloalkyl, a hydroxyl, a thiacycloalkyl-1,1dioxide and a (C₁-C₆)alkyloxy,
         a -NH-heterocycloalkyl, a -NH-cycloalkyl, a -N((C₁-C₆)alkyl)-heterocycloalkyl, or a -NH((C₁-C₆)alkyl)-thiacycloalkyl-1,1dioxide, optionally substituted by a hydroxyl, a (C₁-C₆)alkyl, a (C₁-C₆)alkyloxy or a -CO-R₆ with R₆ being a hydrogen or a (C₁-C₆)alkyl,
         -a hydroxy, a —CN, a -CO-R₆ or a -CO₂R₆ with R₆ being a hydrogen or a (C₁-C₆)alkyl,
         a (C₁-C₆)alkyloxy optionally substituted by at least one radical selected in the group consisting of a halogen, preferably a fluorine, a hydroxy, a (C₁-C₆)alkyloxy, a -NR₇R₈ with R₇ and R₈ are independently a hydrogen or a (C₁-C₆)alkyl, a -NHCOR₉, a -NHCO₂R₉, with R_{g} being a (C₁-C₆)alkyl, a -CO₂R₆ with R₆ being a hydrogen or a (C₁-C₆)alkyl, and a heterocycle,
         a -NHCOR₉, a -NHCO₂R₉, or a -SO₂R₉, with R₉ being a (C₁-C₆)alkyl, and
         a heterocycloalkyl, a bridged heterocycloalkyl, a heterocycloalkyloxy, a cycloalkyloxy, a thiaheterocycloalkyl-1,1-dioxide or a spiroheterocycloalkyl, optionally substituted by a (C₁-C₆)alkyl, a (C₁-C₆)alkyloxy, a hydroxy, a ketone, a halogen or a (C₁-C₆)alkyl optionally substituted by a (C₁-C₆)alkyloxy, or
   a (C₁-C₆)alkyl or a (C₂-C₆)alkenyl, optionally substituted by a 5-10 membered ring as defined above or a -CO₂R₆ with R₆ being a hydrogen or a (C₁-C₆)alkyl;
R₄ represents a —COOH;
R₅ represents:
   a hydrogen, or
   a (C₁-C₆)alkyl optionally substituted by at least one halogen, preferably optionally substituted by at least one fluorine;
and the stereoisomers, and the pharmaceutical salts thereof.

In one aspect, R₁ represents an optionally substituted fused arylcycloalkyl. Preferably, R₁ represents an optionally substituted fused arylcycloalkyl selected in a group consisting of an indanyl, a 1,2,3,4-tetrahydronaphtalenyl, and a 6,7,8,9-tetrahydro-5H-benzo[7]annulenyl, preferably an indanyl and a 1,2,3,4-tetrahydronaphtalenyl, more preferably a 1,2,3,4-tetrahydronaphtalenyl, preferably being

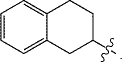

Optionally, R₃ represents an aryl optionally fused to a heterocycloalkyl, preferably selected from the group consisting of a dioxole, a morpholine, a dioxane, a tetrahydropyran, and a tetrahydrofuran, or a heteroaryl, said aryl, fused aryl, or heteroaryl is optionally substituted by at least one radical selected in the group consisting of:
a heterocycloalkyl or a bridged heterocycloalkyl, optionally substituted by a (C₁-C₆)alkyl, a (C₁-C₆)alkyloxy, or a ketone,
a thiaheterocycloalkyl-1,1-dioxide, a heterocycloalkyloxy, or a cycloalkyloxy;
a (C₁-C₆)alkyloxy or a (C₁-C₆)alkyl, optionally substituted by at least one halogen, preferably a fluorine, or a (C₁-C₆)alkyloxy,
a halogen, preferably a fluorine or a chlorine,
a -NH-(C₁-C₆)alkyl or a -N-((C₁-C₆)alkyl)₂, optionally substituted by a heterocycloalkyl, a cycloalkyl, a hydroxyl, a thiacycloalkyl-1,1-dioxide or a (C₁-C₆)alkyloxy,
a -NH-heterocycloalkyl, a -N((C₁-C₆)alkyl)-heterocycloalkyl, or a -NH((C₁-C₆)alkyl)-thiacycloalkyl-1,1-dioxide,
a hydroxy,
a —CN,
a (C₁-C₆)alkyl substituted by an optionally bridged heterocycloalkyl or an optionally substituted heterocycloalkyl; and
a -SO₂R_{9,} with R₉ being a (C₁-C₆)alkyl.

More specifically, R₃ represents a phenyl, a pyridinyl or a pyrimidinyl, preferably a phenyl, optionally substituted by at least one radical selected in the group consisting of:
a morpholinyl optionally substituted by at least one methyl,
a -NH-tetrahydropyranyl,
a -NH-(C₁-C₆)alkyl or a -N(CH₃)( C₁-C₆)alkyl), optionally substituted by a tetrahydropyranyl, a cyclohexyl, an optionally bridged morpholinyl optionally substituted by at least one methyl, a thiacycloalkyl-1,1-dioxide, a hydroxy, or a (C₁-C₆)alkyloxy,
an azetidinyl optionally substituted by a (C₁-C₆)alkyloxy,
a pyrrolidin-2-one,
a 6-oxa-3-azabicyclo[3.1.1]heptane, or a 8 oxa-3-azabicyclo[3.2.1]octane,
a (C₁-C₆)alkyloxy, optionally substituted by at least one halogen, preferably a fluorine, or one (C₁-C₆)alkyloxy,
a halogen, preferably a fluorine and a chlorine,
a hydroxy,
a —CN,
a —SO₂—CH₃,
a 1,1-dioxo-1,2-thiazolidin,
a cyclobutyloxy, or a tetrahydropyranyloxy,
a (C₁-C₆)alkyl optionally substituted by at least one halogen, preferably optionally substituted by at least one fluorine, and
a (C₁-C₆)alkyl substituted by a morpholinyl optionally substituted by at least one methyl, a 6-oxa-3-azabicyclo[3.1.1]heptane, a 8 oxa-3-azabicyclo[3.2.1]octane or a tetrahydropyranyl.

Optionally, R₂ represents
a hydrogen,
a halogen, preferably a chlorine or a fluorine, and
an optionally substituted (C₃-C₆)cycloalkyl, preferably cyclopropyl; preferably a hydrogen, a chlorine or a fluorine.

Optionally, said compound is such as R₁ is

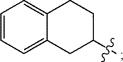

R₂ is a hydrogen or a halogen, preferably a halogen such as F or Cl; R₃ is a phenyl optionally substituted by a halogen such as F or Cl; and R₅ is a hydrogen.

In a particular aspect, said compound is selected in the group consisting of compounds of the table A. In a more particular aspect, said compound is selected in the group consisting of compound #16, compound #17, compound #19, compound #57, compound #86, compound #94, compound #124, compound #140, compound #151, compound #152, compound #162, compound #171, compound #174, compound #175, compound #180, compound #194, compound #195, and compound #196. In a more specific aspect, said compound is selected in the group consisting of compound #16 and compound #157. In a very specific aspect, said compound is compound #157.

In a particular aspect, said compound is selected in the group consisting of compound #16, compound #17, compound #157, compound #195, and compound #196.

In another particular aspect, said compound is selected in the group consisting of compounds of the table B. In a more particular aspect, said compound is selected in the group consisting of compound #B9, compound #B18 and compound #B111.

In a first aspect, the subject has a renal fibrosis and suffers from a chronic kidney disease (CKD). More particularly, the subject may have a CKD at a stage selecting from stage 1, stage 2, stage 3 or stage 4, preferably stage 2, stage 3 or stage 4, more preferably stage 2 or stage 3.

Optionally, the subject having a renal fibrosis suffers from a hypertension, type 2 diabetes, type 1 diabetes, obesity, ageing, infectious glomerulonephritis, focal segmental glomerulosclerosis, IgA nephropathy, minimal change glomerulopathy, membranous nephropathy, renal vasculitis, urinary tract obstruction, genetic alterations, autoimmune diseases such as systemic lupus erythematosus (SLE), and drug- or toxin-induced nephropathy.

In a very specific aspect, the subject has a renal fibrosis and suffers from a disease selected from the group consisting of Non-alcoholic fatty liver disease (NAFLD) and non-alcoholic steatohepatitis (NASH). In this very specific aspect, the compound such as R₁ is

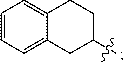

R₂ is a hydrogen or a halogen, preferably a halogen such as F or Cl; R₃ is a phenyl optionally substituted by a halogen such as F or Cl; and R₅ is a hydrogen. Optionally, said compound is selected in the group consisting of compound #16, compound #17, compound #157, compound #195, and compound #196. More particular, said compound is selected in the group consisting of compound #16 and compound #157.

### BRIEF DESCRIPTION OF THE FRIGURES

FIG. 1: Compound #157 treatment significantly decreases renal fibrosis in NASH mice. Mice were fed with an AMLN diet for 30 weeks prior PO treatment with Compound #157 for 8 weeks. Kidneys were collected at sacrifice, and fibrosis was evaluated by quantification of the picrosirius red (PSR) staining positive areas. p-values are indicated on the graph.

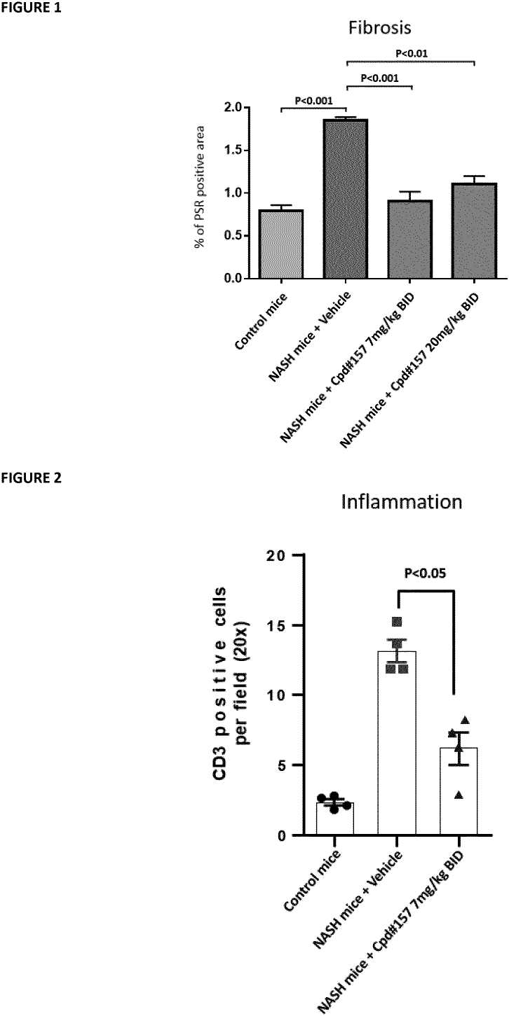

FIG. 2: Compound #157 treatment significantly decreases renal inflammation in NASH mice. Mice were fed with an AMLN diet for 30 weeks prior PO treatment with Compound #157 for 8 weeks. Kidneys were collected at sacrifice, and Inflammation was quantified on CD3 stained sections. p-value is indicated on the graph.

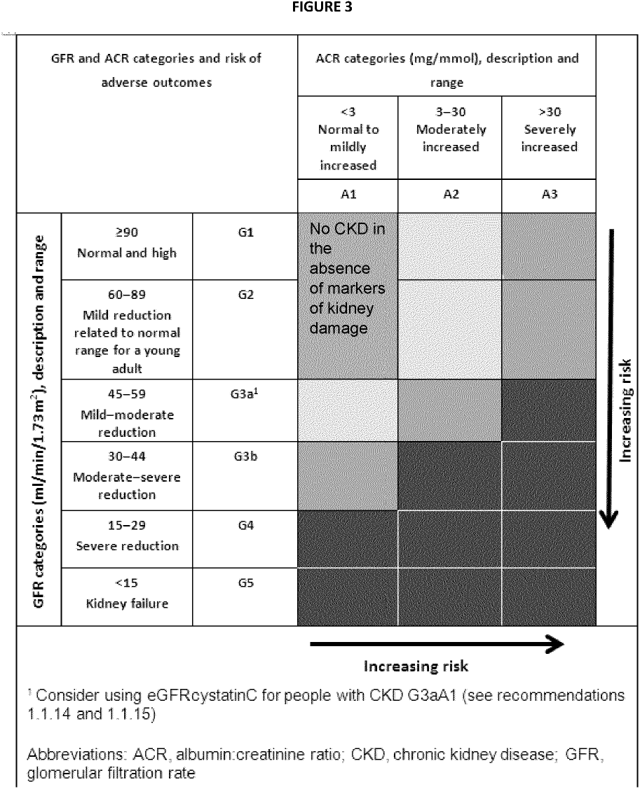

FIG. 3: Chronic kidney disease classification based upon glomerular filtration rate and albuminuria. Data from: KDIGO. Summary of recommendation statements. Kidney Int 2013, 3 (suppl), 5; and National Kidney Foundation. K/DOQI clinical practice guidelines for chronic kidney disease: evaluation, classification, and stratification, Am J Kidney Dis 2002, 39 (Suppl 1): S1.

### DETAILED DESCRIPTION OF THE INVENTION

### Definitions

According to the present invention, the terms below have the following meanings:

The terms mentioned herein with prefixes such as for example C₁-C₃, C₁-C₆ or C₂-C₆ can also be used with lower numbers of carbon atoms such as C₁-C₂, C₁-C₅, or C₂-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₂-C₆ is used, it means that the corresponding hydrocarbon chain may comprise from 2 to 6 carbon atoms, especially 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl, more preferably a methyl.

The term “alkenyl” refers to an unsaturated, linear or branched aliphatic group comprising at least one carbon-carbon double bound. The term “(C₂-C₆)alkenyl” more specifically means ethenyl, propenyl, isopropenyl, butenyl, isobutenyl, pentenyl, or hexenyl.

The term “alkoxy” or “alkyloxy” corresponds to the alkyl group as above defined bonded to the molecule by an —O— (ether) bond. (C₁-C₃)alkoxy includes methoxy, ethoxy, propyloxy, and isopropyloxy. (C₁-C₆)alkoxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl. The term “cycloalkyl” may also refer to a 5-10 membered bridged carbocyclyl such as bicyclo[2,2,1]heptanyl, bicyclo[2,2,2]octanyl, bicyclo[1.1.1]pentanyl, or adamantyl, preferably bicyclo[2,2,1]heptanyl. In a preferred embodiment, the “cycloalkyl” is a cyclopropyl, cyclobutyl, cyclopentyl or a cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or spiro-connected heterocycloalkyl groups. Representative heterocycloalkyl groups include, but are not limited to 3-dioxolane, benzo [1,3] dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl. The term “heterocycloalkyl” may also refer to a 5-10 membered bridged heterocyclyl such as 7-oxabicyclo[2,2,1]heptanyl, 6-oxa-3-azabicyclo[3,1,1]heptanyl, and 8-oxa-3-azabicyclo[3,1,1]octanyl. In a particular embodiment, it may also refer to spiro-connected heterocycloalkyl groups or spiroheterocycloalkyl groups such as for instance oxetanyl spiro-connected with azetidinyl or piperidinyl. In a preferred embodiment, the heterocycloalkyl group is azetidinyl, oxetanyl, pyranyl, tetrahydrofuranyl, tetrahydropyranyl, morpholinyl, piperidinyl, piperazinyl, and oxetanyl spiro-connected with azetidinyl or piperidinyl.

The term “aryl” corresponds to a mono- or bi-cyclic aromatic hydrocarbons having from 6 to 12 carbon atoms. For instance, the term “aryl” includes phenyl, biphenyl, or naphthyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolinyl, benzothienyl, benzothiazolyl, isatinyl, dihydropyridyl, pyrimidinyl, s-triazinyl, oxazolyl, or thiofuranyl. In a preferred embodiment, the heteroaryl group is a pyridinyl, furanyl, pyrazolyl, pyrazinyl, pyrimidinyl, pyridazinyl, and isoxazolyl.

The terms “fused arylheterocycloalkyl” and “fused arylcycloalkyl” correspond to a bicyclic group in which an aryl as above defined is bounded to the heterocycloalkyl or the cycloalkyl as above defined by at least two carbons. In other terms, the aryl shares a carbon bond with the heterocycloalkyl or the cycloalkyl. A fused arylheterocycloalkyl is for instance a benzodioxole (phenyl fused to a dioxole), an isobenzofurane or a benzomorpholine (phenyl fused to a morpholine. A fused arylcycloalkyl is for instance an indanyl, a 1,2,3,4-tetrahydronaphtalenyl (also called tetralinyl), or a 6,7,8,9-tetrahydro-5H-benzo[7]annulenyl (fused phenyl-C₇-cycloalkyl). The term “fused bicycloalkyl” corresponds to a bicyclic group in which a cycloalkyl as above defined is bounded to the cycloalkyl as above defined by at least two carbons. A fused bicycloalkyl is for instance a bicyclo[4.1.0]heptanyl.

The term “halogen” corresponds to a fluorine, chlorine, bromine, or iodine atom, preferably a fluorine, chlorine or bromine.

The expression “substituted by at least” means that the radical is substituted by one or several groups of the list.

The expression “optionally substituted” means, without any otherwise precision, optionally substituted by a hydroxy, a halogen, a (C₁-C₆)alkyl optionally substituted by at least one halogen, preferably optionally substituted by at least one fluorine, or a (C₁-C₆)alkoxy optionally substituted by at least one halogen, preferably optionally substituted by at least one fluorine.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred embodiment of the invention, the stereoisomers include diastereoisomers and enantiomers. The enantiomers compounds may be prepared from the racemate compound using any purification method known by a skilled person, such as LC/MS and chiral HPLC analysis methods and chiral SFC purification methods such as those disclosed in the examples (Example A - Chemistry, Table 1 and Table 3).

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is selected from the group consisting of sodium and potassium salt.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

### Compounds

The compounds of the present invention can be any compound disclosed in WO 2019/154956 and WO 2019/154953, the disclosure of which being incorporated herein by reference.

In a first aspect, the compounds of the present invention can be any compound disclosed in WO 2019/154956, in particular any compound disclosed in Table A. In a second aspect, the compounds of the present invention can be any compound disclosed in WO 2019/154953, in particular any compound disclosed in Table B.

In a particular aspect, the compounds of the present invention have the formula (I)

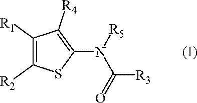

wherein:
R₁ represents a fused arylcycloalkyl or a cycloalkyl optionally substituted by at least one radical selected in the group consisting of:
   a halogen,
   a (C₁-C₆)alkyl or a (C₁-C₆)alkyloxy optionally substituted by at least one halogen, preferably optionally substituted by at least one fluorine, and
   a hydroxy,
   a -CO-R₆ or a -CO₂R₆ with R₆ being a hydrogen or a (C₁-C₆)alkyl, and
   an optionally substituted aryl;
➢ R₂ represents:
   a hydrogen,
   a halogen,
   a (C₁-C₆)alkyl optionally substituted by at least one halogen, preferably optionally substituted by at least one fluorine,
   an optionally substituted aryl, or
   an optionally substituted cycloalkyl;
➢ R₃ represents:
   a 5-10 membered ring, saturated or unsaturated selected in the group consisting of:
      an aryl optionally fused to a heterocycloalkyl, preferably selected from the group consisting of a dioxole, a morpholine, a dioxane, a tetrahydropyran, and a tetrahydrofuran,
      a heteroaryl,
      a cycloalkyl,
      a heterocycloalkyl, and
      a 5-10 membered bridged carbocyclyl or heterocyclyl, said 5-10 membered ring is optionally substituted by at least one radical selected in the group consisting of:
         a halogen,
         a (C₁-C₆)alkyl optionally substituted by at least one halogen, preferably optionally substituted by at least one fluorine, or by an optionally bridged heterocycloalkyl optionally substituted by a (C₁-C₆)alkyl,
         a -NH-(C₁-C₆)alkyl or a -N-((C₁-C₆)alkyl)₂, optionally substituted by at least one radical selected in the group consisting of a heterocycloalkyl, a cycloalkyl, a hydroxyl, a thiacycloalkyl-1,1dioxide and a (C₁-C₆)alkyloxy,
         a -NH-heterocycloalkyl, a -NH-cycloalkyl, a -N((C₁-C₆)alkyl)-heterocycloalkyl, or a -NH((C₁-C₆)alkyl)-thiacycloalkyl-1,1dioxide, optionally substituted by a hydroxyl, a (C₁-C₆)alkyl, a (C₁-C₆)alkyloxy or a -CO-R₆ with R₆ being a hydrogen or a (C₁-C₆)alkyl,
         a hydroxy, a —CN, a -CO-R₆ or a -CO₂R₆ with R₆ being a hydrogen or a (C₁-C₆)alkyl,
         a (C₁-C₆)alkyloxy optionally substituted by at least one radical selected in the group consisting of a halogen, preferably a fluorine, a hydroxy, a (C₁-C₆)alkyloxy, a -NR₇Rₛ with R₇ and R₈ are independently a hydrogen or a (C₁-C₆)alkyl, a -NHCOR₉, a -NHCO₂R₉, with R₉ being a (C₁-C₆)alkyl, a -CO₂R₆ with R₆ being a hydrogen or a (C₁-C₆)alkyl, and a heterocycle,
         a -NHCOR₉, a -NHCO₂R₉, or a -SO₂R₉, with R₉ being a (C₁-C₆)alkyl, and
         a heterocycloalkyl, a bridged heterocycloalkyl, a heterocycloalkyloxy, a cycloalkyloxy, a thiaheterocycloalkyl-1,1-dioxide or a spiroheterocycloalkyl, optionally substituted by a (C₁-C₆)alkyl, a (C₁-C₆)alkyloxy, a hydroxy, a ketone, a halogen or a (C₁-C₆)alkyl optionally substituted by a (C₁-C₆)alkyloxy, or
   a (C₁-C₆)alkyl or a (C₂-C₆)alkenyl, optionally substituted by a 5-10 membered ring as defined above or a -CO₂R₆ with R₆ being a hydrogen or a (C₁-C₆)alkyl;
➢ R₄ represents a —COOH;
➢ R₅ represents:
   a hydrogen, or
   a (C₁-C₆)alkyl optionally substituted by at least one halogen, preferably optionally substituted by at least one fluorine;
and the stereoisomers, and the pharmaceutical salts thereof.

Preferably, R₂ represents a hydrogen, a halogen, preferably a chlorine or a fluorine, and an optionally substituted (C₃-C₆)cycloalkyl, preferably cyclopropyl. In a preferred aspect, R₂ represents a hydrogen, a chlorine or a fluorine. In a specific aspect, R₂ is a hydrogen. In another specific aspect, R₂ is a chlorine or a fluorine.

Preferably, R₃ represents an aryl optionally fused to a heterocycloalkyl, preferably selected from the group consisting of a dioxole, a morpholine, a dioxane, a tetrahydropyran, and a tetrahydrofuran, or a heteroaryl, said aryl, fused aryl, or heteroaryl is optionally substituted by at least one radical selected in the group consisting of:
a heterocycloalkyl or a bridged heterocycloalkyl, optionally substituted by a (C₁-C₆)alkyl, a (C₁-C₆)alkyloxy, or a ketone,
a thiaheterocycloalkyl-1,1-dioxide, a heterocycloalkyloxy, or a cycloalkyloxy;
a (C₁-C₆)alkyloxy or a (C₁-C₆)alkyl, optionally substituted by at least one halogen, preferably a fluorine, or a (C₁-C₆)alkyloxy,
a halogen, preferably a fluorine or a chlorine,
a -NH-(C₁-C₆)alkyl or a -N-((C₁-C₆)alkyl)₂, optionally substituted by a heterocycloalkyl, a cycloalkyl, a hydroxyl, a thiacycloalkyl-1,1-dioxide or a (C₁-C₆)alkyloxy,
a -NH-heterocycloalkyl, a -N((C₁-C₆)alkyl)-heterocycloalkyl, or a -NH((C₁-C₆)alkyl)-thiacycloalkyl-1,1-dioxide,
a hydroxy,
a —CN,
a (C₁-C₆)alkyl substituted by an optionally bridged heterocycloalkyl or an optionally substituted heterocycloalkyl; and
a -SO₂R₉, with R₉ being a (C₁-C₆)alkyl.

More particularly, R₃ represents a phenyl, a pyridinyl or a pyrimidinyl, preferably a phenyl, optionally substituted by at least one radical selected in the group consisting of:
a morpholinyl optionally substituted by at least one methyl,
a -NH-tetrahydropyranyl,
a -NH-(C₁-C₆)alkyl or a -N(CH₃)(C₁-C₆)alkyl), optionally substituted by a tetrahydropyranyl, a cyclohexyl, an optionally bridged morpholinyl optionally substituted by at least one methyl, a thiacycloalkyl-1,1-dioxide, a hydroxy, or a (C₁-C₆)alkyloxy,
an azetidinyl optionally substituted by a (C₁-C₆)alkyloxy,
a pyrrolidin-2-one,
a 6-oxa-3-azabicyclo[3.1.1]heptane, or a 8 oxa-3-azabicyclo[3.2.1]octane,
a (C₁-C₆)alkyloxy, optionally substituted by at least one halogen, preferably a fluorine, or one (C₁-C₆)alkyloxy,
a halogen, preferably a fluorine and a chlorine,
a hydroxy,
a —CN,
a —SO₂—CH₃,
a 1,1-dioxo-1,2-thiazolidin,
a cyclobutyloxy, or a tetrahydropyranyloxy,
a (C₁-C₆)alkyl optionally substituted by at least one halogen, preferably optionally substituted by at least one fluorine, and
a (C₁-C₆)alkyl substituted by a morpholinyl optionally substituted by at least one methyl, a 6-oxa-3-azabicyclo[3.1.1]heptane, a 8 oxa-3-azabicyclo[3.2.1]octane or a tetrahydropyranyl.

In a particular aspect, R₃ is a radical selected in the group consisting of:

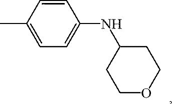

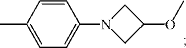

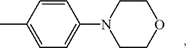

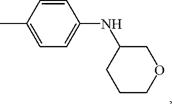

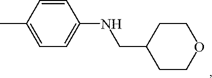

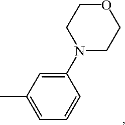

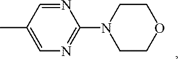

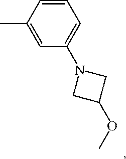

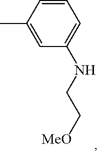

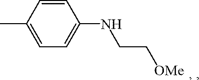

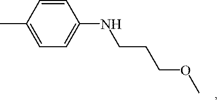

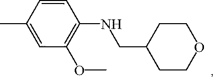

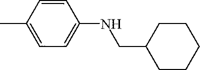

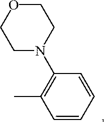

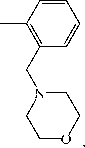

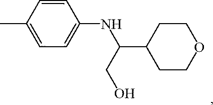

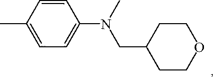

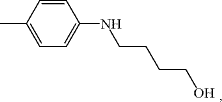

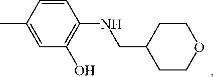

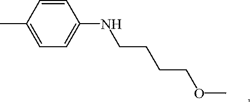

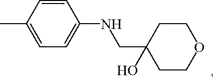

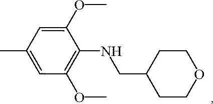

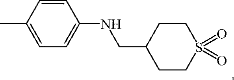

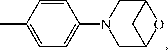

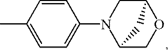

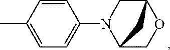

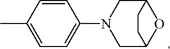

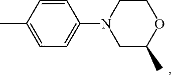

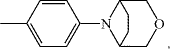

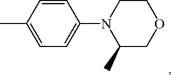

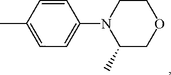

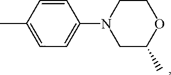

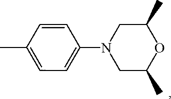

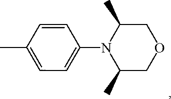

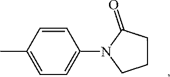

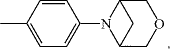

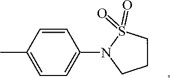

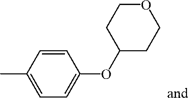

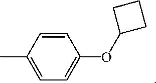

In another particular aspect, R₃ is a radical selected in the group consisting of:

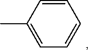

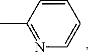

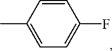

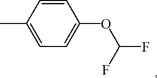

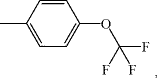

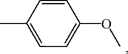

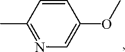

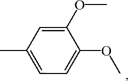

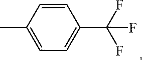

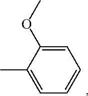

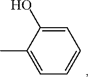

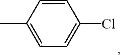

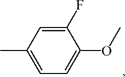

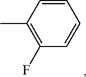

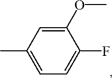

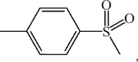

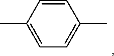

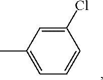

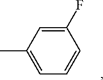

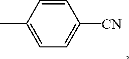

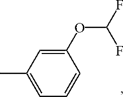

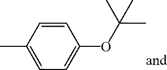

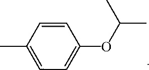

In a further particular aspect, R3 is a radical selected in the group consisting of:

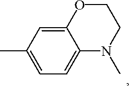

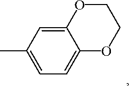

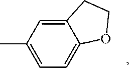

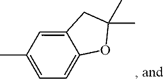

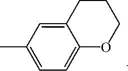

In a very specific aspect, R₃ represents a phenyl, optionally substituted by a halogen, preferably a fluorine. In this aspect, R₃ can be

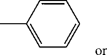

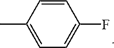

In a particular aspect, R₂ represents a halogen, preferably a fluorine or a chlorine, and R₃ can be

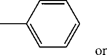

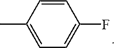

In the first aspect, R₁ represents an optionally substituted fused arylcycloalkyl. More specifically, R₁ represents an optionally substituted fused arylcycloalkyl selected in a group consisting of an indanyl, a 1,2,3,4-tetrahydronaphtalenyl, and a 6,7,8,9-tetrahydro-5H-benzo[7]annulenyl, preferably an indanyl and a 1,2,3,4-tetrahydronaphtalenyl, more preferably a 1,2,3,4-tetrahydronaphtalenyl.

For instance, the optionally substituted fused arylcycloalkyl R₁ can comprise a radical selected in a group consisting of:

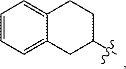

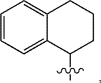

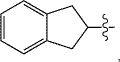

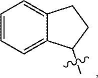

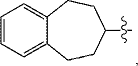

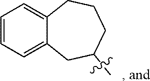

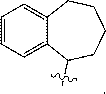

Alternatively, the optionally substituted fused arylcycloalkyl R₁ can comprise a radical selected in a group consisting of:

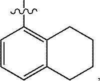

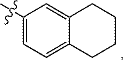

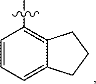

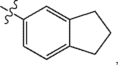

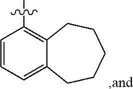

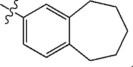

In a particular aspect, R₁ is

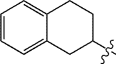

or a substituted radical thereof.

In a very specific aspect of the disclosure, R₁ represents

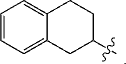

In a particular embodiment, as R₁ is

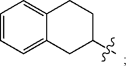

R₂ is a hydrogen or a halogen, preferably a halogen such as For Cl; R₃ is a phenyl optionally substituted by a halogen such as F or Cl; and R₅ is a hydrogen.

In a preferred embodiment, the compound according to the present invention is selected in the group consisting of compounds of the table A below:

TABLE A

| | | |
|---|---|---|
| 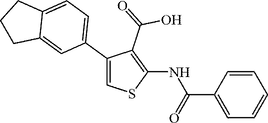 [SMILES: O=C(Nc1scc(-c2ccc3c(c2)CCC3)c1C(=O)O)c1ccccc1] | 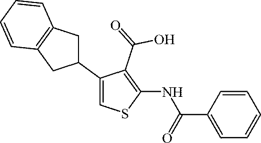 [SMILES: O=C(Nc1scc(C2Cc3ccccc3C2)c1C(=O)O)c1ccccc1] | 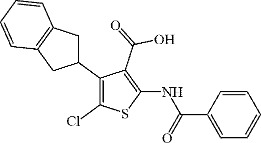 [SMILES: O=C(Nc1sc(Cl)c(C2Cc3ccccc3C2)c1C(=O)O)c1ccccc1] |
| Compound #3 | Compound #7 | Compound #8 |
| 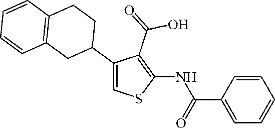 [SMILES: O=C(Nc1scc(C2CCc3ccccc3C2)c1C(=O)O)c1ccccc1] | 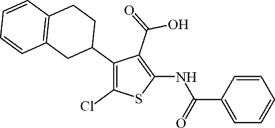 [SMILES: O=C(Nc1sc(Cl)c(C2CCc3ccccc3C2)c1C(=O)O)c1ccccc1] | 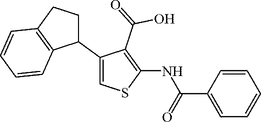 [SMILES: O=C(Nc1scc(C2CCc3ccccc32)c1C(=O)O)c1ccccc1] |
| Compound #16 | Compound #17 | Compound #19 |
| 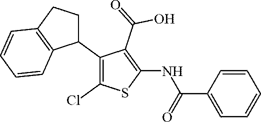 [SMILES: O=C(Nc1sc(Cl)c(C2CCc3ccccc32)c1C(=O)O)c1ccccc1] | 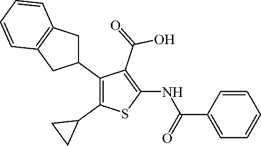 [SMILES: O=C(Nc1sc(C2CC2)c(C2Cc3ccccc3C2)c1C(=O)O)c1ccccc1] | 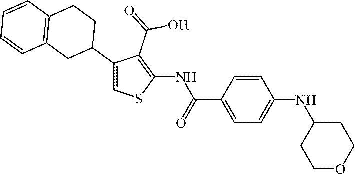 [SMILES: O=C(Nc1scc(C2CCc3ccccc3C2)c1C(=O)O)c1ccc(NC2CCOCC2)cc1] |
| Compound #49 | Compound #51 | Compound #52 |
| 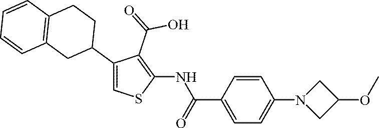 [SMILES: COC1CN(c2ccc(C(=O)Nc3scc(C4CCc5ccccc5C4)c3C(=O)O)cc2)C1] | 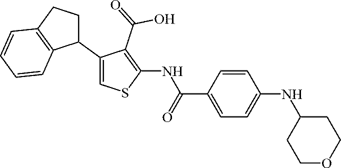 [SMILES: O=C(Nc1scc(C2CCc3ccccc32)c1C(=O)O)c1ccc(NC2CCOCC2)cc1] | 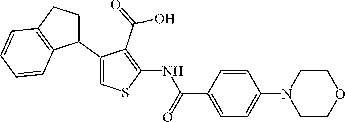 [SMILES: O=C(Nc1scc(C2CCc3ccccc32)c1C(=O)O)c1ccc(N2CCOCC2)cc1] |
| Compound #53 | Compound #55 | Compound #56 |
| 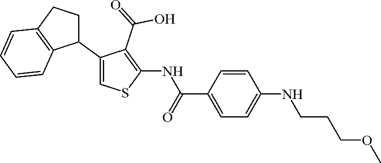 [SMILES: COCCCNc1ccc(C(=O)Nc2scc(C3CCc4ccccc43)c2C(=O)O)cc1] | 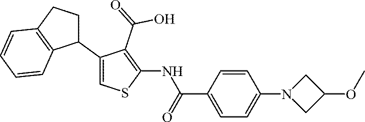 [SMILES: COC1CN(c2ccc(C(=O)Nc3scc(C4CCc5ccccc54)c3C(=O)O)cc2)C1] | 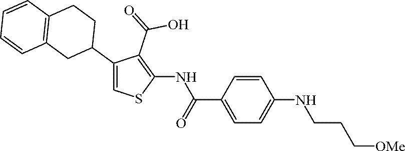 [SMILES: COCCCNc1ccc(C(=O)Nc2scc(C3CCc4ccccc4C3)c2C(=O)O)cc1] |
| Compound #57 | Compound #58 | Compound #64 |
| 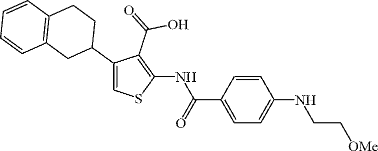 [SMILES: COCCNc1ccc(C(=O)Nc2scc(C3CCc4ccccc4C3)c2C(=O)O)cc1] | 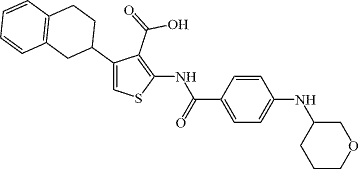 [SMILES: O=C(Nc1scc(C2CCc3ccccc3C2)c1C(=O)O)c1ccc(NC2CCCOC2)cc1] | 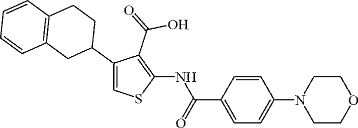 [SMILES: O=C(Nc1scc(C2CCc3ccccc3C2)c1C(=O)O)c1ccc(N2CCOCC2)cc1] |
| Compound #65 | Compound #67 | Compound #68 |
| 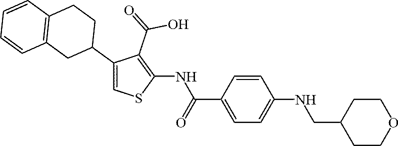 [SMILES: O=C(Nc1scc(C2CCc3ccccc3C2)c1C(=O)O)c1ccc(NCC2CCOCC2)cc1] | 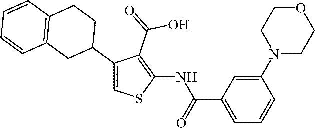 [SMILES: O=C(Nc1scc(C2CCc3ccccc3C2)c1C(=O)O)c1cccc(N2CCOCC2)c1] | 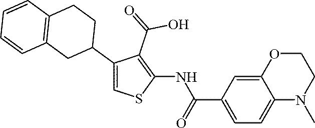 [SMILES: CN1CCOc2cc(C(=O)Nc3scc(C4CCc5ccccc5C4)c3C(=O)O)ccc21] |
| Compound #69 | Compound #70 | Compound #86 |
| 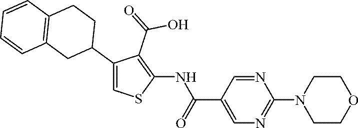 [SMILES: O=C(Nc1scc(C2CCc3ccccc3C2)c1C(=O)O)c1cnc(N2CCOCC2)nc1] | 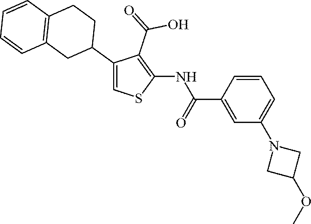 [SMILES: COC1CN(c2cccc(C(=O)Nc3scc(C4CCc5ccccc5C4)c3C(=O)O)c2)C1] | 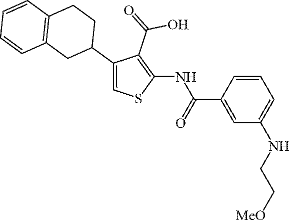 [SMILES: COCCNc1cccc(C(=O)Nc2scc(C3CCc4ccccc4C3)c2C(=O)O)c1] |
| Compound #89 | Compound #93 | Compound #94 |
| 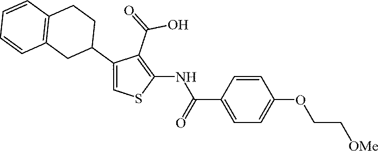 [SMILES: COCCOc1ccc(C(=O)Nc2scc(C3CCc4ccccc4C3)c2C(=O)O)cc1] | 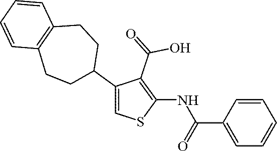 [SMILES: O=C(Nc1scc(C2CCc3ccccc3CC2)c1C(=O)O)c1ccccc1] | 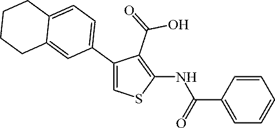 [SMILES: O=C(Nc1scc(-c2ccc3c(c2)CCCC3)c1C(=O)O)c1ccccc1] |
| Compound #101 | | Compound #123 |
| | Compound #122 | |
| 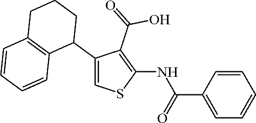 [SMILES: O=C(Nc1scc(C2CCCc3ccccc32)c1C(=O)O)c1ccccc1] | 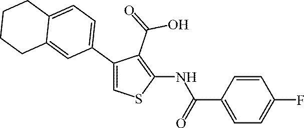 [SMILES: O=C(Nc1scc(-c2ccc3c(c2)CCCC3)c1C(=O)O)c1ccc(F)cc1] | 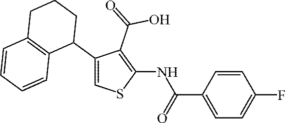 [SMILES: O=C(Nc1scc(C2CCCc3ccccc32)c1C(=O)O)c1ccc(F)cc1] |
| | Compound #125 | Compound #126 |
| Compound #124 | | |
| 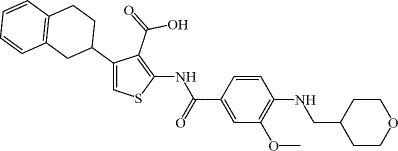 [SMILES: COc1cc(C(=O)Nc2scc(C3CCc4ccccc4C3)c2C(=O)O)ccc1NCC1CCOCC1] | 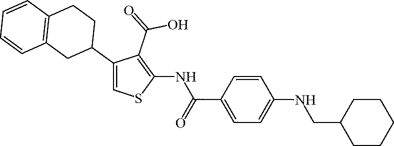 [SMILES: O=C(Nc1scc(C2CCc3ccccc3C2)c1C(=O)O)c1ccc(NCC2CCCCC2)cc1] | 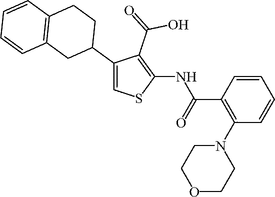 [SMILES: O=C(Nc1scc(C2CCc3ccccc3C2)c1C(=O)O)c1ccccc1N1CCOCC1] |
| Compound #127 | Compound #128 | Compound #129 |
| 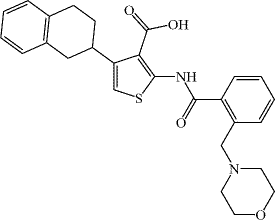 [SMILES: O=C(Nc1scc(C2CCc3ccccc3C2)c1C(=O)O)c1ccccc1CN1CCOCC1] | 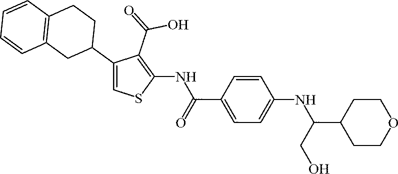 [SMILES: O=C(Nc1scc(C2CCc3ccccc3C2)c1C(=O)O)c1ccc(NC(CO)C2CCOCC2)cc1] | 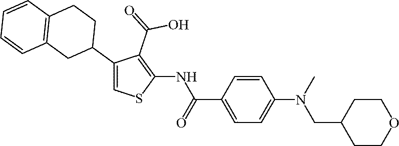 [SMILES: CN(CC1CCOCC1)c1ccc(C(=O)Nc2scc(C3CCc4ccccc4C3)c2C(=O)O)cc1] |
| Compound #130 | Compound #131 | Compound #132 |
| 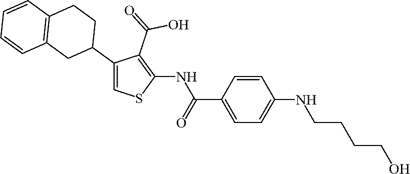 [SMILES: O=C(Nc1scc(C2CCc3ccccc3C2)c1C(=O)O)c1ccc(NCCCCO)cc1] | 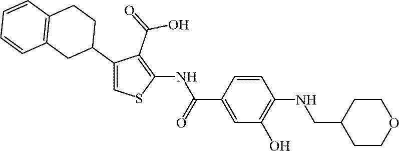 [SMILES: O=C(Nc1scc(C2CCc3ccccc3C2)c1C(=O)O)c1ccc(NCC2CCOCC2)c(O)c1] | 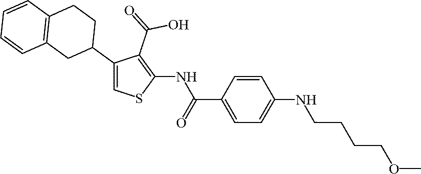 [SMILES: COCCCCNc1ccc(C(=O)Nc2scc(C3CCc4ccccc4C3)c2C(=O)O)cc1] |
| Compound #133 | Compound #134 | Compound #135 |
| 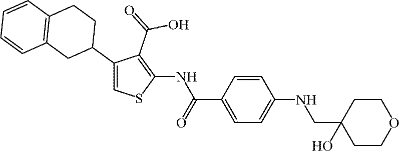 [SMILES: O=C(Nc1scc(C2CCc3ccccc3C2)c1C(=O)O)c1ccc(NCC2(O)CCOCC2)cc1] | 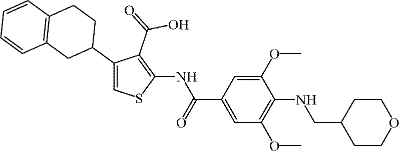 [SMILES: COc1cc(C(=O)Nc2scc(C3CCc4ccccc4C3)c2C(=O)O)cc(OC)c1NCC1CCOCC1] | 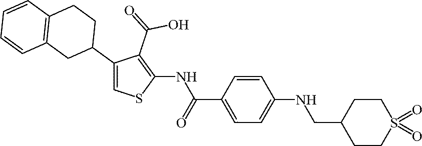 [SMILES: O=C(Nc1scc(C2CCc3ccccc3C2)c1C(=O)O)c1ccc(NCC2CCS(=O)(=O)CC2)cc1] |
| Compound #136 | Compound #137 | Compound #138 |
| 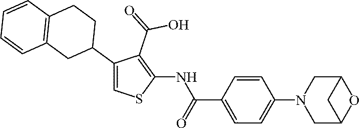 [SMILES: O=C(Nc1scc(C2CCc3ccccc3C2)c1C(=O)O)c1ccc(N2CC3CC(C2)O3)cc1] | 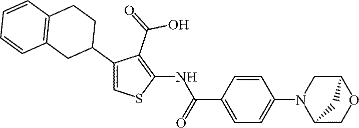 [SMILES: O=C(Nc1scc(C2CCc3ccccc3C2)c1C(=O)O)c1ccc(N2C[C@@H]3C[C@H]2CO3)cc1] | 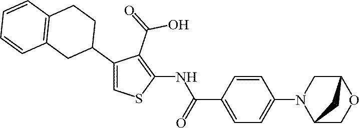 [SMILES: O=C(Nc1scc(C2CCc3ccccc3C2)c1C(=O)O)c1ccc(N2C[C@H]3C[C@@H]2CO3)cc1] |
| Compound #139 | Compound #140 | Compound #141 |
| 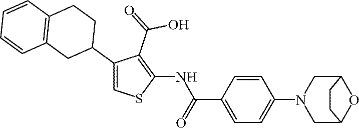 [SMILES: O=C(Nc1scc(C2CCc3ccccc3C2)c1C(=O)O)c1ccc(N2CC3CCC(C2)O3)cc1] | 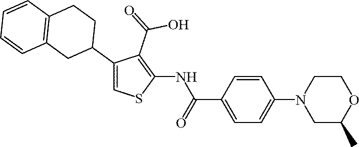 [SMILES: C[C@H]1CN(c2ccc(C(=O)Nc3scc(C4CCc5ccccc5C4)c3C(=O)O)cc2)CCO1] | 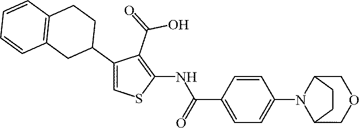 [SMILES: O=C(Nc1scc(C2CCc3ccccc3C2)c1C(=O)O)c1ccc(N2C3CCC2COC3)cc1] |
| Compound #142 | Compound #143 | Compound #144 |
| 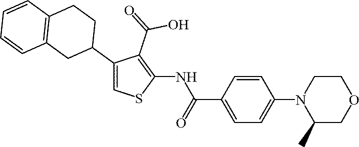 [SMILES: C[C@@H]1COCCN1c1ccc(C(=O)Nc2scc(C3CCc4ccccc4C3)c2C(=O)O)cc1] | 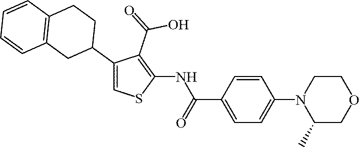 [SMILES: C[C@H]1COCCN1c1ccc(C(=O)Nc2scc(C3CCc4ccccc4C3)c2C(=O)O)cc1] | 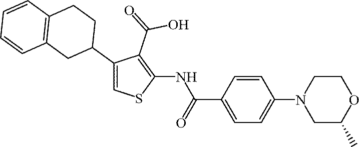 [SMILES: C[C@@H]1CN(c2ccc(C(=O)Nc3scc(C4CCc5ccccc5C4)c3C(=O)O)cc2)CCO1] |
| Compound #145 | Compound #146 | Compound #147 |
| 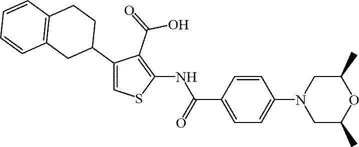 [SMILES: C[C@@H]1CN(c2ccc(C(=O)Nc3scc(C4CCc5ccccc5C4)c3C(=O)O)cc2)C[C@H](C)O1] | 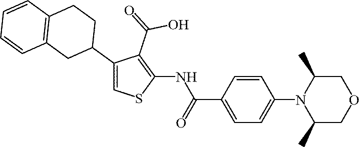 [SMILES: C[C@@H]1COC[C@H](C)N1c1ccc(C(=O)Nc2scc(C3CCc4ccccc4C3)c2C(=O)O)cc1] | 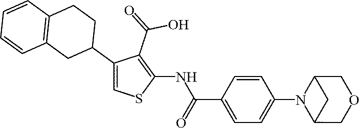 [SMILES: O=C(Nc1scc(C2CCc3ccccc3C2)c1C(=O)O)c1ccc(N2C3COCC2C3)cc1] |
| Compound #148 | Compound #149 | Compound #150 |
| 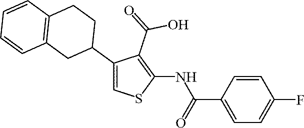 [SMILES: O=C(Nc1scc(C2CCc3ccccc3C2)c1C(=O)O)c1ccc(F)cc1] | 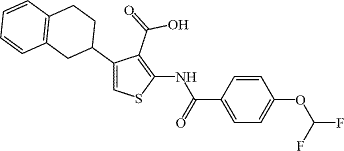 [SMILES: O=C(Nc1scc(C2CCc3ccccc3C2)c1C(=O)O)c1ccc(OC(F)F)cc1] | 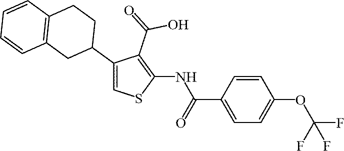 [SMILES: O=C(Nc1scc(C2CCc3ccccc3C2)c1C(=O)O)c1ccc(OC(F)(F)F)cc1] |
| Compound #151 | Compound #152 | Compound #153 |
| 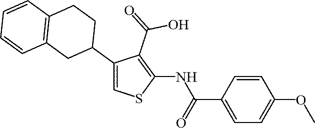 [SMILES: COc1ccc(C(=O)Nc2scc(C3CCc4ccccc4C3)c2C(=O)O)cc1] | 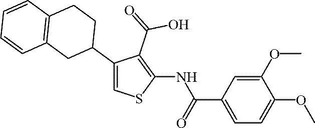 [SMILES: COc1ccc(C(=O)Nc2scc(C3CCc4ccccc4C3)c2C(=O)O)cc1OC] | 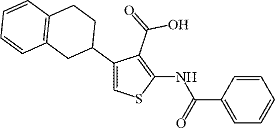 [SMILES: O=C(Nc1scc(C2CCc3ccccc3C2)c1C(=O)O)c1ccccc1] |
| Compound #154 | Compound #155 | Compound #156 |
| 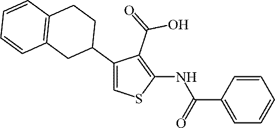 [SMILES: O=C(Nc1scc(C2CCc3ccccc3C2)c1C(=O)O)c1ccccc1] | 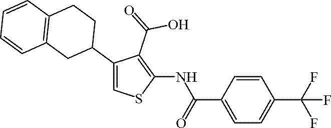 [SMILES: O=C(Nc1scc(C2CCc3ccccc3C2)c1C(=O)O)c1ccc(C(F)(F)F)cc1] | 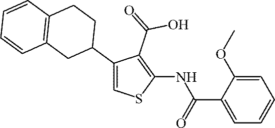 [SMILES: COc1ccccc1C(=O)Nc1scc(C2CCc3ccccc3C2)c1C(=O)O] |
| Compound #157 | Compound #158 | Compound #159 |
| 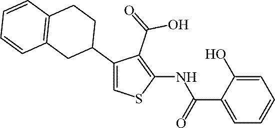 [SMILES: O=C(Nc1scc(C2CCc3ccccc3C2)c1C(=O)O)c1ccccc1O] | 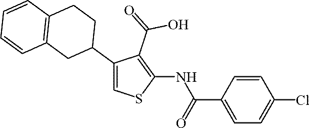 [SMILES: O=C(Nc1scc(C2CCc3ccccc3C2)c1C(=O)O)c1ccc(Cl)cc1] | 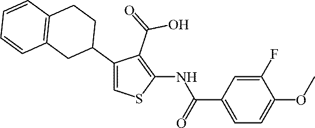 [SMILES: COc1ccc(C(=O)Nc2scc(C3CCc4ccccc4C3)c2C(=O)O)cc1F] |
| Compound #160 | Compound #161 | Compound #162 |
| 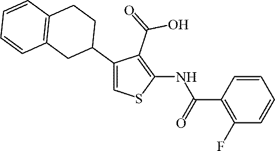 [SMILES: O=C(Nc1scc(C2CCc3ccccc3C2)c1C(=O)O)c1ccccc1F] | 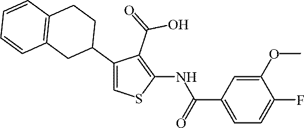 [SMILES: COc1cc(C(=O)Nc2scc(C3CCc4ccccc4C3)c2C(=O)O)ccc1F] | 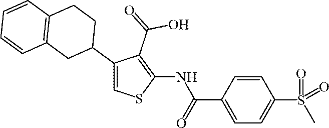 [SMILES: CS(=O)(=O)c1ccc(C(=O)Nc2scc(C3CCc4ccccc4C3)c2C(=O)O)cc1] |
| Compound #163 | Compound #164 | Compound #165 |
| 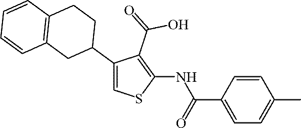 [SMILES: Cc1ccc(C(=O)Nc2scc(C3CCc4ccccc4C3)c2C(=O)O)cc1] | 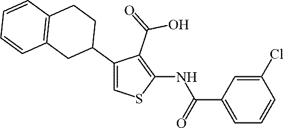 [SMILES: O=C(Nc1scc(C2CCc3ccccc3C2)c1C(=O)O)c1cccc(Cl)c1] | 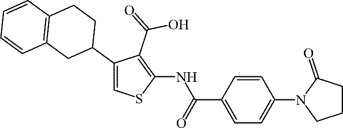 [SMILES: O=C(Nc1scc(C2CCc3ccccc3C2)c1C(=O)O)c1ccc(N2CCCC2=O)cc1] |
| Compound #166 | Compound #167 | Compound #168 |
| 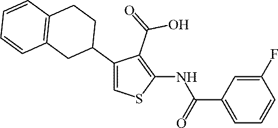 [SMILES: O=C(Nc1scc(C2CCc3ccccc3C2)c1C(=O)O)c1cccc(F)c1] | 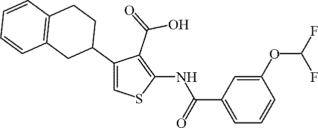 [SMILES: O=C(Nc1scc(C2CCc3ccccc3C2)c1C(=O)O)c1cccc(OC(F)F)c1] | 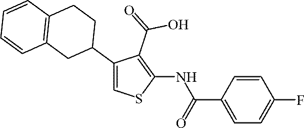 [SMILES: O=C(Nc1scc(C2CCc3ccccc3C2)c1C(=O)O)c1ccc(F)cc1] |
| Compound #169 | Compound #170 | Compound #171 |
| 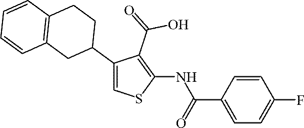 [SMILES: O=C(Nc1scc(C2CCc3ccccc3C2)c1C(=O)O)c1ccc(F)cc1] | 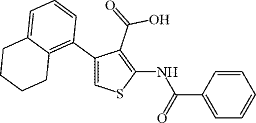 [SMILES: O=C(Nc1scc(-c2cccc3c2CCCC3)c1C(=O)O)c1ccccc1] | 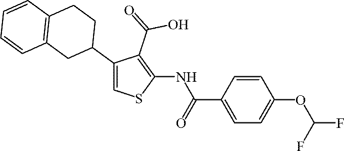 [SMILES: O=C(Nc1scc(C2CCc3ccccc3C2)c1C(=O)O)c1ccc(OC(F)F)cc1] |
| Compound #172 | | Compound #174 |
| 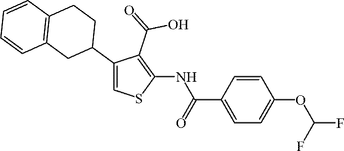 [SMILES: O=C(Nc1scc(C2CCc3ccccc3C2)c1C(=O)O)c1ccc(OC(F)F)cc1] | 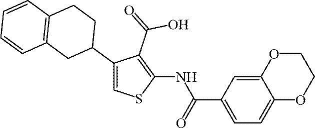 [SMILES: O=C(Nc1scc(C2CCc3ccccc3C2)c1C(=O)O)c1ccc2c(c1)OCCO2] | 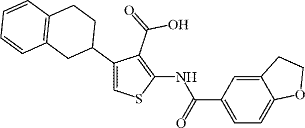 [SMILES: O=C(Nc1scc(C2CCc3ccccc3C2)c1C(=O)O)c1ccc2c(c1)CCO2] |
| Compound #175 | Compound #176 | Compound #177 |
| 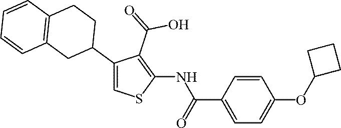 [SMILES: O=C(Nc1scc(C2CCc3ccccc3C2)c1C(=O)O)c1ccc(OC2CCC2)cc1] | 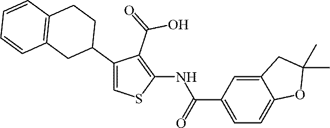 [SMILES: CC1(C)Cc2cc(C(=O)Nc3scc(C4CCc5ccccc5C4)c3C(=O)O)ccc2O1] | 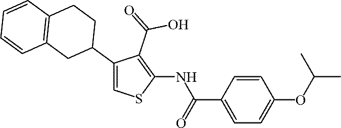 [SMILES: CC(C)Oc1ccc(C(=O)Nc2scc(C3CCc4ccccc4C3)c2C(=O)O)cc1] |
| Compound #178 | Compound #179 | Compound #180 |
| 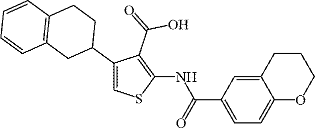 [SMILES: O=C(Nc1scc(C2CCc3ccccc3C2)c1C(=O)O)c1ccc2c(c1)CCCO2] | 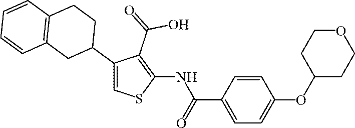 [SMILES: O=C(Nc1scc(C2CCc3ccccc3C2)c1C(=O)O)c1ccc(OC2CCOCC2)cc1] | 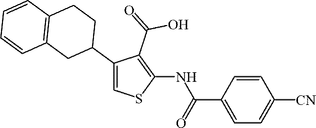 [SMILES: N#Cc1ccc(C(=O)Nc2scc(C3CCc4ccccc4C3)c2C(=O)O)cc1] |
| Compound #181 | Compound #182 | Compound #183 |
| 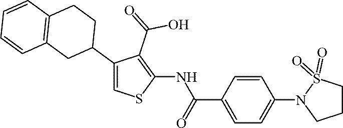 [SMILES: O=C(Nc1scc(C2CCc3ccccc3C2)c1C(=O)O)c1ccc(N2CCCS2(=O)=O)cc1] Compound #184 | 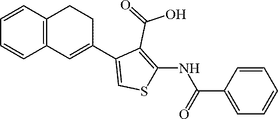 [SMILES: O=C(Nc1scc(C2=Cc3ccccc3CC2)c1C(=O)O)c1ccccc1] | 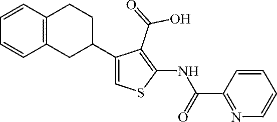 [SMILES: O=C(Nc1scc(C2CCc3ccccc3C2)c1C(=O)O)c1ccccn1] |
| | Compound #185 | Compound #186 |
| 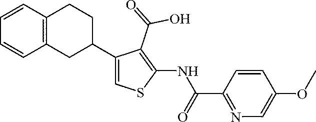 [SMILES: COc1ccc(C(=O)Nc2scc(C3CCc4ccccc4C3)c2C(=O)O)nc1] | 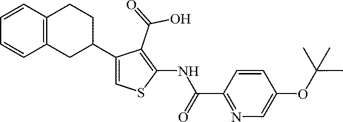 [SMILES: CC(C)(C)Oc1ccc(C(=O)Nc2scc(C3CCc4ccccc4C3)c2C(=O)O)nc1] | 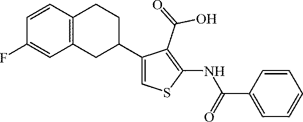 [SMILES: O=C(Nc1scc(C2CCc3ccc(F)cc3C2)c1C(=O)O)c1ccccc1] |
| Compound #187 | Compound #188 | Compound #189 |
| 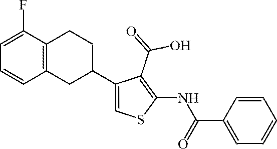 [SMILES: O=C(Nc1scc(C2CCc3c(F)cccc3C2)c1C(=O)O)c1ccccc1] | 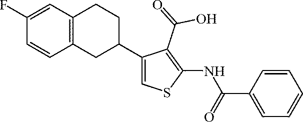 [SMILES: O=C(Nc1scc(C2CCc3cc(F)ccc3C2)c1C(=O)O)c1ccccc1] | 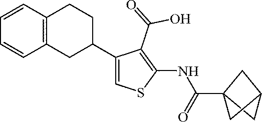 [SMILES: O=C(O)c1c(C2CCc3ccccc3C2)csc1NC(=O)C12CC(C1)C2] |
| Compound #190 | Compound #191 | Compound #192 |
| 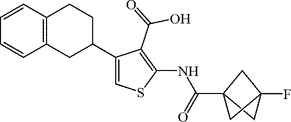 [SMILES: O=C(O)c1c(C2CCc3ccccc3C2)csc1NC(=O)C12CC(F)(C1)C2] | 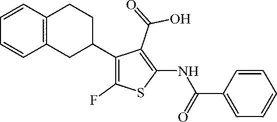 [SMILES: O=C(Nc1sc(F)c(C2CCc3ccccc3C2)c1C(=O)O)c1ccccc1] | 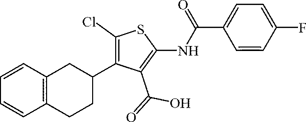 [SMILES: O=C(Nc1sc(Cl)c(C2CCc3ccccc3C2)c1C(=O)O)c1ccc(F)cc1] |
| Compound #193 | Compound #194 | Compound #195 |
| 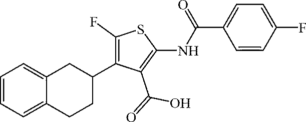 [SMILES: O=C(Nc1sc(F)c(C2CCc3ccccc3C2)c1C(=O)O)c1ccc(F)cc1] | | |
| Compound #196 | | |

In a very particular aspect, the compound is selected from the group consisting of compound #16, compound #17, compound #19, compound #57, compound #86, compound #94, compound #124, compound #140, compound #151, compound #152, compound #157, compound #162, compound #171, compound #174, compound #175, compound #194, compound #195 and compound #196. In a particular aspect, the compound is selected from the group consisting of compound #16 and compound #157. In a very particular aspect, the compound is compound #157.

In another very particular aspect, the compound is selected from the group consisting of compound #16, compound #17, compound #157, compound #195, and compound #196. More specifically, the compound is selected from the group consisting of compound #16 and compound #157.

In the second aspect, R₁ represents an optionally substituted cycloalkyl.

In a particular aspect, R₁ is a radical selected in the group consisting of:

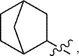

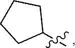

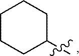

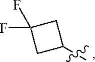

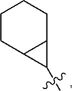

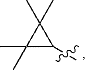

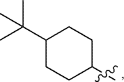

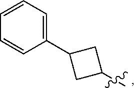

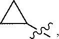

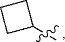

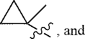

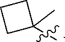

In a preferred aspect, the compound is selected in the group consisting of compounds of the table B below:

TABLE B

| | | |
|---|---|---|
| 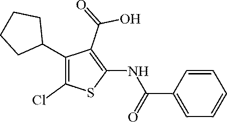 [SMILES: O=C(Nc1sc(Cl)c(C2CCCC2)c1C(=O)O)c1ccccc1] | 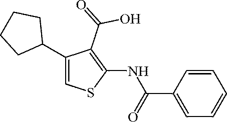 [SMILES: O=C(Nc1scc(C2CCCC2)c1C(=O)O)c1ccccc1] | 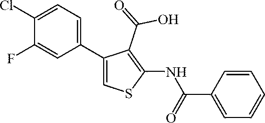 [SMILES: O=C(Nc1scc(-c2ccc(Cl)c(F)c2)c1C(=O)O)c1ccccc1] |
| Compound #B18 | Compound #B10 | Compound #B2 |
| 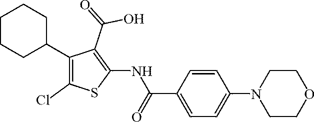 [SMILES: O=C(Nc1sc(Cl)c(C2CCCCC2)c1C(=O)O)c1ccc(N2CCOCC2)cc1] | 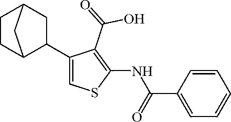 [SMILES: O=C(Nc1scc(C2CC3CCC2C3)c1C(=O)O)c1ccccc1] | 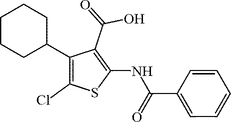 [SMILES: O=C(Nc1sc(Cl)c(C2CCCCC2)c1C(=O)O)c1ccccc1] |
| Compound #B15 | Compound #B9 | Compound #B12 |
| 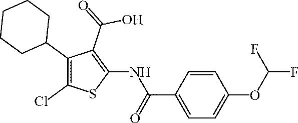 [SMILES: O=C(Nc1sc(Cl)c(C2CCCCC2)c1C(=O)O)c1ccc(OC(F)F)cc1] | 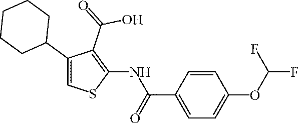 [SMILES: O=C(Nc1scc(C2CCCCC2)c1C(=O)O)c1ccc(OC(F)F)cc1] | 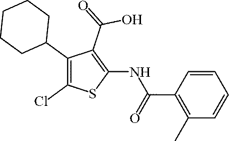 [SMILES: Cc1ccccc1C(=O)Nc1sc(Cl)c(C2CCCCC2)c1C(=O)O] |
| Compound #B13 | Compound #B11 | |
| | | Compound #B14 |
| 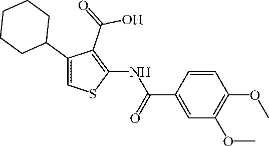 [SMILES: COc1ccc(C(=O)Nc2scc(C3CCCCC3)c2C(=O)O)cc1OC] | 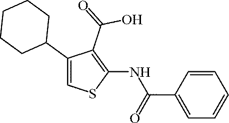 [SMILES: O=C(Nc1scc(C2CCCCC2)c1C(=O)O)c1ccccc1] | 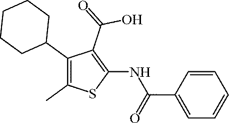 [SMILES: Cc1sc(NC(=O)c2ccccc2)c(C(=O)O)c1C1CCCCC1] |
| Compound #B25 | Compound #B26 | Compound #B27 |
| 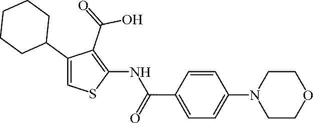 [SMILES: O=C(Nc1scc(C2CCCCC2)c1C(=O)O)c1ccc(N2CCOCC2)cc1] | 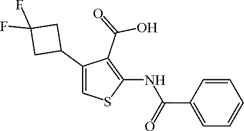 [SMILES: O=C(Nc1scc(C2CC(F)(F)C2)c1C(=O)O)c1ccccc1] | 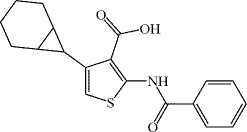 [SMILES: O=C(Nc1scc(C2C3CCCCC32)c1C(=O)O)c1ccccc1] |
| Compound #B29 | Compound #B35 | Compound #B37 |
| 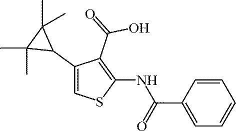 [SMILES: CC1(C)C(c2csc(NC(=O)c3ccccc3)c2C(=O)O)C1(C)C] | 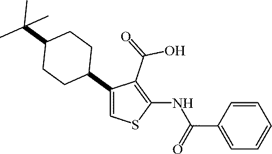 [SMILES: CC(C)(C)[C@H]1CC[C@@H](c2csc(NC(=O)c3ccccc3)c2C(=O)O)CC1] | 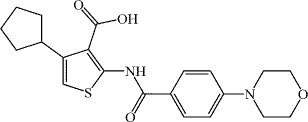 [SMILES: O=C(Nc1scc(C2CCCC2)c1C(=O)O)c1ccc(N2CCOCC2)cc1] |
| Compound #B39 | | Compound #B59 |
| | Compound #B54 | |
| 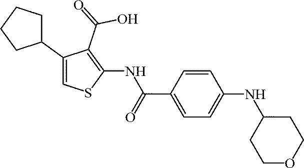 [SMILES: O=C(Nc1scc(C2CCCC2)c1C(=O)O)c1ccc(NC2CCOCC2)cc1] | 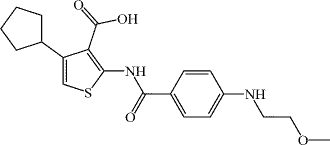 [SMILES: COCCNc1ccc(C(=O)Nc2scc(C3CCCC3)c2C(=O)O)cc1] | 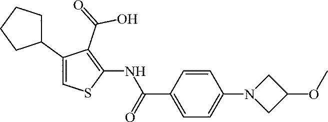 [SMILES: COC1CN(c2ccc(C(=O)Nc3scc(C4CCCC4)c3C(=O)O)cc2)C1] |
| Compound #B60 | Compound #B61 | Compound #B62 |
| 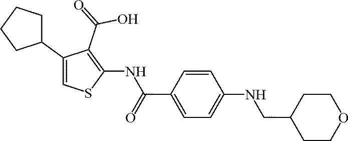 [SMILES: O=C(Nc1scc(C2CCCC2)c1C(=O)O)c1ccc(NCC2CCOCC2)cc1] | 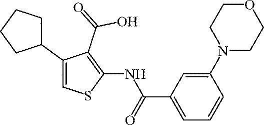 [SMILES: O=C(Nc1scc(C2CCCC2)c1C(=O)O)c1cccc(N2CCOCC2)c1] | 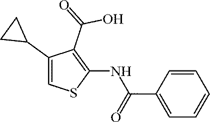 [SMILES: O=C(Nc1scc(C2CC2)c1C(=O)O)c1ccccc1] |
| Compound #B66 | Compound #B71 | Compound #B87 |
| 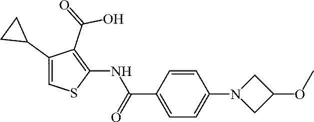 [SMILES: COC1CN(c2ccc(C(=O)Nc3scc(C4CC4)c3C(=O)O)cc2)C1] | 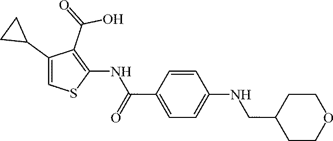 [SMILES: O=C(Nc1scc(C2CC2)c1C(=O)O)c1ccc(NCC2CCOCC2)cc1] | 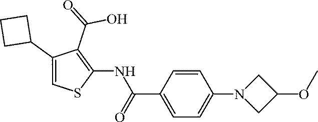 [SMILES: COC1CN(c2ccc(C(=O)Nc3scc(C4CCC4)c3C(=O)O)cc2)C1] |
| Compound #B88 | Compound #B90 | Compound #B97 |
| 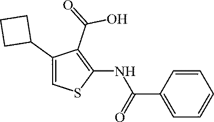 [SMILES: O=C(Nc1scc(C2CCC2)c1C(=O)O)c1ccccc1] | 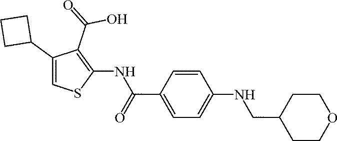 [SMILES: O=C(Nc1scc(C2CCC2)c1C(=O)O)c1ccc(NCC2CCOCC2)cc1] | 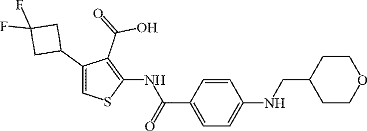 [SMILES: O=C(Nc1scc(C2CC(F)(F)C2)c1C(=O)O)c1ccc(NCC2CCOCC2)cc1] |
| Compound #B98 | Compound #B99 | Compound #B106 |
| 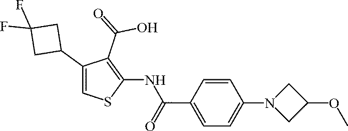 [SMILES: COC1CN(c2ccc(C(=O)Nc3scc(C4CC(F)(F)C4)c3C(=O)O)cc2)C1] | 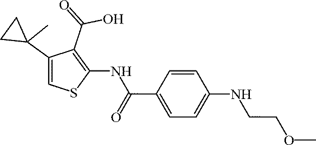 [SMILES: COCCNc1ccc(C(=O)Nc2scc(C3(C)CC3)c2C(=O)O)cc1] | 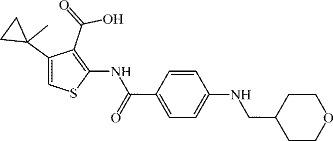 [SMILES: CC1(c2csc(NC(=O)c3ccc(NCC4CCOCC4)cc3)c2C(=O)O)CC1] |
| Compound #B107 | | Compound #B111 |
| | Compound #B108 | |
| 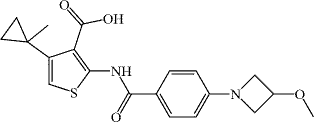 [SMILES: COC1CN(c2ccc(C(=O)Nc3scc(C4(C)CC4)c3C(=O)O)cc2)C1] | 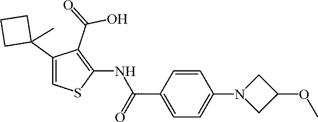 [SMILES: COC1CN(c2ccc(C(=O)Nc3scc(C4(C)CCC4)c3C(=O)O)cc2)C1] | |
| Compound #B112 | Compound #B115 | |

In a very particular aspect, the compound is selected from the group consisting of compound #B9, compound #B18 and compound #B111.

The present invention relates to the use of any one of these compounds.

### Diseases

The compounds of the present invention show a specific effect on renal fibrosis. Indeed, in the animal model, the compounds of the invention completely resolve renal fibrosis and attenuate significantly renal inflammation.

In a particular aspect, the compounds of the present invention are able to decrease the renal fibrosis. For example, the decrease could be of 20, 30, 40, 50, 60, 70, 80, 90 or 100% in comparison of the renal fibrosis in absence of treatment with the compound. The decrease can be measured by any method available for the person skilled in the art, for instance in an animal model as detailed in the example. Optionally, the renal fibrosis is a glomerulosclerosis. Optionally, the renal fibrosis is more particularly a glomerulosclerosis. Optionally, the renal fibrosis is more particularly a tubulointerstitial fibrosis. Optionally, the renal fibrosis is both glomerulosclerosis and tubulointerstitial fibrosis.

In addition, he compounds of the present invention are able to decrease the renal inflammation. For example, the decrease could be of 10, 20, 30, 40, or 50% in comparison of the renal inflammation in absence of treatment with the compound. The decrease can be measured by any method available for the person skilled in the art, for instance in an animal model as detailed in the example.

The subject has a renal fibrosis. The renal fibrosis can be diagnosed based on a kidney biopsy. Alternatively, it can be diagnosed based on an alternative analysis such as magnetic resonance imaging (MRI) or urinary tract proteomics (e.g., CKD273)

In a particular aspect, the renal fibrosis can include glomerulosclerosis. In another particular aspect, the renal fibrosis can include tubulointerstitial fibrosis. In an additional particular aspect, the renal fibrosis can include both glomerulosclerosis and tubulointerstitial fibrosis.

In a particular aspect, the subject has a renal fibrosis and suffers from a chronic kidney disease. Chronic Kidney Disease (CKD) is defined as the presence of kidney damage (usually detected as urinary albumin excretion of ≥30 mg/day or equivalent) or decreased kidney function (defined as estimated glomerular filtration rate [eGFR] <60 mL/min/1.73 m2) for three or more months, irrespective of the cause.

In a particular aspect, the subject has a renal fibrosis and has a CKD stage chosen from G1, G2, G3a, G3b, G4 or G5, preferably G1, G2, G3a, G3b, or G4 based upon glomerular filtration rate (eGFR) as indicated in FIG. 3, more preferably G2, G3a, G3b, or G4, still more particularly G2, G3a, or G3b.

In another particular aspect, the subject has a renal fibrosis and has a CKD stage chosen from A1, A2 or A3 based upon albuminuria (ACR) as indicated in FIG. 3.

TABLE 1

Staging of CKD

| Staging | | ACR | | |
|---|---|---|---|---|
| | | A1 | A2 | A3 |
| GFR | G1 | 1* if kidney damage present | 1 | 2 |
| | G2 | 1* if kidney damage present | 1 | 2 |
| | G3a | 1 | 2 | 3 |
| | G3b | 2 | 3 | 3 |
| | G4 | 3 | 4+ | 4+ |
| | G5 | 4+ | 4+ | 4+ |

In a first particular aspect, the subject has a renal fibrosis and has a CKD of stage 1* defined as of G1 or G2 based upon eGFR as indicated in FIG. 3 and of A1 based upon ACR as indicated in FIG. 3.

In a second particular aspect, the subject has a renal fibrosis and has a CKD of stage 1 defined as of G1 or G2 based upon eGFR as indicated in FIG. 3 and of A2 based upon ACR as indicated in FIG. 3 or of G3a based upon eGFR and of A1 based upon ACR.

In a third particular aspect, the subject has a renal fibrosis and has a CKD of stage 2 defined as of G1 or G2 based upon eGFR as indicated in FIG. 3 and of A3 based upon ACR as indicated in FIG. 3 or of G3a based upon eGFR and of A2 based upon ACR, or of G3b based upon eGFR and of A1 based upon ACR.

In a fourth particular aspect, the subject has a renal fibrosis and has a CKD of stage 3 defined as of G3a based upon eGFR as indicated in FIG. 3 and of A3 based upon ACR as indicated in FIG. 3 or of G3b based upon eGFR and of A2 or A3 based upon ACR, or of G4 based upon eGFR and of A based upon ACR.

The effect of the compound on the disease can be for instance assessed by the measure of eGFR or ACR. A therapeutic effect could be the improvement of eGFR and/or ACR. The therapeutic effect could be the stabilization of eGFR and/or ACR. The therapeutic effect could also be to delay the progression of the disease or to slow down the progression of the disease, for instance as assessed by eGFR and/or ACR assays.

In a particular aspect, the subject has a renal fibrosis and suffers from a disease selected from the group consisting of hypertension, type 2 diabetes, type 1 diabetes, obesity, ageing, infectious glomerulonephritis, in particular infections such as syphilis, malaria, hepatitis B, hepatitis C or HIV, focal segmental glomerulosclerosis, IgA nephropathy, minimal change glomerulopathy, membranous nephropathy, renal vasculitis, urinary tract obstruction, genetic alterations, autoimmune diseases such as systemic lupus erythematosus (SLE), and drug- or toxin-induced nephropathy such as nephropathy induced by drugs such as captopril, NSAIDs, penicillamine, probenecid, bucillamine, anti-TNF therapy, and tiopronin or by toxins such as inorganic salts (e.g., gold, mercury).

In another particular aspect, the subject has a renal fibrosis and suffers from a primary kidney disease such as focal segmental glomerulosclerosis, IgA nephropathy, minimal change glomerulopathy, membranous nephropathy, urinary-tract infections, stones, obstruction diseases, and renal vasculitis.

In another particular aspect, the subject has a renal fibrosis and suffers from a systemic disease affecting the kidney, for instance a disease selected from the group consisting of hypertension, type 2 diabetes, type 1 diabetes, infectious glomerulonephritis, in particular infections such as syphilis, malaria, hepatitis B, hepatitis C or HIV, renal vasculitis, autoimmune diseases such as systemic lupus erythematosus (SLE), and drug- or toxin-induced nephropathy such as neuphropathy induced by drugs such as captopril, NSAIDs, penicillamine, probenecid, bucillamine, anti-TNF therapy, and tiopronin or by toxins such as inorganic salts (e.g., gold, mercury).

Optionally, the subject has a renal fibrosis and suffers from hypertension. The subject may have a CKD of stage 1, 2 or 3 as defined above.

Optionally, the subject has a renal fibrosis and suffers from type 2 diabetes or type 1 diabetes. The subject may have a CKD of stage 1,2 or 3 as defined above.

Optionally, the subject has a renal fibrosis and suffers from systemic lupus erythematosus (SLE). The subject may have a CKD of stage 1, 2 or 3 as defined above. In this context, the subject may have a lupus nephropathy of class III, IV, V or VI.

Optionally, the subject has a renal fibrosis and suffers from NASH or NAFLD. The subject may have a CKD of stage 1, 2 or 3 as defined above. Non-alcoholic steatohepatitis (NASH) is a disease characterized by excessive fat accumulation, inflammation, and ballooning degeneration of hepatocytes, with or without fibrosis in the liver. In addition, some subjects affected by NASH may further present chronic kidney disease. For these particular subjects, the compounds of the invention could be of particular interest. Indeed, the compounds of the invention are able to decrease significantly the inflammation and fibrosis in a dose-dependent manner in the liver and they are further capable of significantly inhibiting fibrosis in the kidney and even reversing the existing fibrosis and also of decreasing renal inflammation. Accordingly, the compounds of the present invention could be useful for protecting a subject suffering from NASH of liver and renal lesions or for treating a subject suffering from NASH so as to limit, slow down or reverse liver and renal lesions. Then, the subject is in particular a subject suffering from NASH or NAFLD and having a renal fibrosis. Optionally, the subject is a subject suffering from NASH or NAFLD and from CKD.

### Combinations

The compounds of the present disclosure can be used in combination with other therapeutic agents. The additional therapeutic agents can be selected from the agents already used for the treatment of one of the diseases as specified above. In particular, the additional therapeutic agent could be an anti-inflammatory agent.

### Pharmaceutical Composition

The pharmaceutical composition comprises a compound of the present invention and optionally at least one pharmaceutically acceptable carrier or excipient.

The compound according to the present disclosure or the pharmaceutical composition according to the present disclosure may be administered by any conventional route of administration. In particular, the compound or the pharmaceutical composition of the present disclosure can be administered by a topical, enteral, oral, parenteral, intranasal, intravenous, intra-arterial, intramuscular, subcutaneous or intraocular administration and the like.

In particular, the compound according to the present disclosure or the pharmaceutical composition according to the present disclosure can be formulated for a topical, enteral, oral, parenteral, intranasal, intravenous, intra-arterial, intramuscular, subcutaneous or intraocular administration and the like.

Preferably, the compound according to the invention or the pharmaceutical composition according to the present disclosure is administered by enteral or parenteral route of administration. When administered parenterally, the compound according to the present disclosure or the pharmaceutical composition according to the present disclosure is preferably administered by intravenous route of administration. When administered enterally, the compound according to the present disclosure or the pharmaceutical composition according to the present disclosure is preferably administered by oral route of administration.

The pharmaceutical composition comprising the molecule is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

The compound according to the invention or the pharmaceutical composition according to the present disclosure may be administered as a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered daily, optionally 1, 2 or 3 times a day.

The duration of treatment with the compound according to the invention or the pharmaceutical composition according to the invention can be weeks, months or even years. In particular, the duration of treatment may last as long as the disease persists.

The amount of compound according to the present disclosure or of pharmaceutical composition according to the present disclosure to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a particular aspect, the total compound dose for each administration of the compound according to the present disclosure or of the pharmaceutical composition according to the present disclosure is comprised between 0.00001 and 1 g.

The form of the pharmaceutical compositions, the route of administration and the dose of administration of the compound according to the present disclosure, or the pharmaceutical composition according to the present disclosure can be adjusted by those skilled in the art according to the type and severity of the disease, and to the patient, in particular its age, weight, sex, and general physical condition.

Further aspects and advantages of the present invention will be described in the following examples, which should be regarded as illustrative and not limiting.

### EXAMPLES

Non-alcoholic steatohepatitis (NASH) is a disease characterized by excessive fat accumulation, inflammation, and ballooning degeneration of hepatocytes, with or without fibrosis in the liver. It is now reported that NASH not only affects the liver but is also associated with kidney dysfunction.

Compound #157 was evaluated in a mouse model of NASH. Mice were fed with an AMLN diet (high in fat (40%), fructose (22%) and cholesterol (2%)) for 30 weeks prior treatment with Compound #157 for 8 weeks twice a day at a dose of 7 or 20 mg/kg.

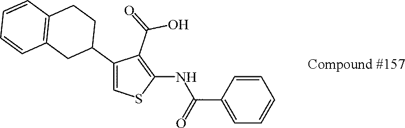 [SMILES: O=C(Nc1scc(C2CCc3ccccc3C2)c1C(=O)O)c1ccccc1]

The livers and kidneys were collected at sacrifice. Fibrosis, in particular tubulointerstitial fibrosis, has been evaluated by quantification of the picrosirius red (PSR) staining positive areas. Inflammation has been quantified on CD3 stained sections.

NASH mice presented not only liver lesions such as steatosis, inflammation, and fibrosis, but also severe renal lesions such as glomerulosclerosis, tubular casts and atrophy, tubular lipid accumulation and interstitial fibrosis. As shown on FIGS. 1 and 2, in the kidneys, Compound #157 was able to completely resolve fibrosis (highly significant effect) and attenuate significantly inflammation (50% decrease), in particular lymphocytes infiltration.

1-19. (canceled)

20. A method of treating a subject having renal fibrosis comprising administering a compound of formula (I), stereoisomers, or pharmaceutical salts thereof or a pharmaceutical composition comprising said compound, stereoisomers, or pharmaceutical salts thereof and an excipient or pharmaceutically acceptable carrier to said subject, said compound for formula (I) having the structure: 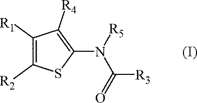
wherein:
R₁ represents a fused arylcycloalkyl or a cycloalkyl optionally substituted by at least one radical selected from the group consisting of:
a halogen,
a (C₁-C₆)alkyl or a (C₁-C₆)alkyloxy optionally substituted by at least one halogen, and
a hydroxy,
a -CO-R₆ or a -CO₂R₆ with R₆ being a hydrogen or a (C₁-C₆)alkyl, and an optionally substituted aryl;
R₂ represents:
a hydrogen,
a halogen,
a (C₁-C₆)alkyl optionally substituted by at least one halogen,
an optionally substituted aryl, or
an optionally substituted cycloalkyl;
R₃ represents:
a 5-10 membered ring, saturated or unsaturated selected from the group consisting of:
an aryl optionally fused to a heterocycloalkyl,
a heteroaryl,
a cycloalkyl,
a heterocycloalkyl, and
a 5-10 membered bridged carbocyclyl or heterocyclyl,
said 5-10 membered ring is optionally substituted by at least one radical selected from the group consisting of:
a halogen,
a (C₁-C₆)alkyl optionally substituted by at least one halogen or by an optionally bridged heterocycloalkyl optionally substituted by a (C₁-C₆)alkyl,
a -NH-(C₁-C₆)alkyl or a -N-((C₁-C₆)alkyl)₂, optionally substituted by at least one radical selected from the group consisting of a heterocycloalkyl, a cycloalkyl, a hydroxyl, a thiacycloalkyl-1, 1dioxide and a (C₁-C₆)alkyloxy,
a -NH-heterocycloalkyl, a -NH-cycloalkyl, a -N((C₁-C₆)alkyl)-heterocycloalkyl, or a -NH((C₁-C₆)alkyl)-thiacycloalkyl-1,1dioxide, optionally substituted by a hydroxyl, a (C₁-C₆)alkyl, a (C₁-C₆)alkyloxy or a CO-R₆ with R₆ being a hydrogen or a (C₁-C₆)alkyl,
a hydroxy, a —CN, a -CO-R₆ or a -CO₂R₆ with R₆ being a hydrogen or a (C₁-C₆)alkyl,
a (C₁-C₆)alkyloxy optionally substituted by at least one radical selected from the group consisting of a halogen, a hydroxy, a (C₁-C₆)alkyloxy, a -NR₇R₈ with R₇ and R₈ are independently a hydrogen or a (C₁-C₆)alkyl, a -NHCOR₉, a -NHCO₂R₉, with R₉ being a (C₁-C₆)alkyl, a -CO₂R₆ with R₆ being a hydrogen or a (C₁-C₆)alkyl, and a heterocycle,
a -NHCOR₉, a -NHCO₂R₉, or a -SO₂R₉, with R₉ being a (C₁-C₆)alkyl, and
a heterocycloalkyl, a bridged heterocycloalkyl, a heterocycloalkyloxy, a cycloalkyloxy, a thiaheterocycloalkyl-1,1-dioxide or a spiroheterocycloalkyl, optionally substituted by a (C₁-C₆)alkyl, a (C₁-C₆)alkyloxy, a hydroxy, a ketone, a halogen or a (C₁-C₆)alkyl optionally substituted by a (C₁-C₆)alkyloxy, or
a (C₁-C₆)alkyl or a (C₂-C₆)alkenyl, optionally substituted by a 5-10 membered ring as defined above or a -CO₂R₆ with R₆ being a hydrogen or a (C₁-C₆)alkyl;
R₄ represents a —COOH;
R₅ represents:
a hydrogen, or
a (C₁-C₆)alkyl optionally substituted by at least one halogen.

21. The method according to claim 20, wherein the renal fibrosis is tubulointerstitial fibrosis.

22. The method according to claim 20, wherein the subject has a renal fibrosis and suffers from a chronic kidney disease (CKD).

23. The method according to claim 22, wherein the CKD has a stage selected from stage 1*, stage 1, stage 2 or stage 3 as defined in Table 1.

24. The method according to claim 20, wherein the subject suffers from a hypertension, type 2 diabetes, type 1 diabetes, obesity, ageing, infectious glomerulonephritis, focal segmental glomerulosclerosis, IgA nephropathy, minimal change glomerulopathy, membranous nephropathy, renal vasculitis, urinary tract obstruction, genetic alterations, systemic lupus erythematosus (SLE), or a drug- or toxin-induced nephropathy.

25. The method according to claim 20, wherein the subject has a renal fibrosis and suffers from a disease selected from the group consisting of Non-alcoholic fatty liver disease (NAFLD) and non-alcoholic steatohepatitis (NASH).

26. The method according to claim 20, wherein R₁ represents an optionally substituted fused arylcycloalkyl.

27. The method according to claim 20, wherein R₁ represents an optionally substituted fused arylcycloalkyl selected from the group consisting of an indanyl, a 1,2,3,4-tetrahydronaphtalenyl, and a 6,7,8,9-tetrahydro-5H-benzo[7]annulenyl, and a 1,2,3,4-tetrahydronaphtalenyl.

28. The method according to claim 20, wherein R₃ represents an aryl optionally fused to a heterocycloalkyl or a heteroaryl, said aryl, fused aryl, or heteroaryl being optionally substituted by at least one radical selected from the group consisting of:
a heterocycloalkyl or a bridged heterocycloalkyl, optionally substituted by a (C₁-C₆)alkyl;
a (C₁-C₆)alkyloxy or a ketone;
a thiaheterocycloalkyl-1,1-dioxide, a heterocycloalkyloxy, or a cycloalkyloxy;
a (C₁-C₆)alkyloxy or a (C₁-C₆)alkyl, optionally substituted by at least one halogen, or a (C₁-C₆)alkyloxy;
a halogen;
a -NH-(C₁-C₆)alkyl or a -N-((C₁-C₆)alkyl)₂, optionally substituted by a heterocycloalkyl, a cycloalkyl, a hydroxyl, a thiacycloalkyl-1,1-dioxide or a (C₁-C₆)alkyloxy;
a -NH-heterocycloalkyl, a -N((C₁-C₆)alkyl)-heterocycloalkyl, or a -NH((C₁-C₆)alkyl)-thiacycloalkyl-1,1-dioxide;
a hydroxy;
a —CN;
a (C₁-C₆)alkyl substituted by an optionally bridged heterocycloalkyl or an optionally substituted heterocycloalkyl; and
a —SO₂R₉, with R₉ being a (C₁-C₆)alkyl.

29. The method according to claim 20, wherein R₃ represents a phenyl, a pyridinyl or a pyrimidinyl, optionally substituted by at least one radical selected from the group consisting of:
a morpholinyl optionally substituted by at least one methyl;
a -NH-tetrahydropyranyl;
a -NH-(C₁-C₆)alkyl or a -N(CH₃)( C₁-C₆)alkyl), optionally substituted by a tetrahydropyranyl, a cyclohexyl, an optionally bridged morpholinyl optionally substituted by at least one methyl, a thiacycloalkyl-1,1-dioxide, a hydroxy, or a (C₁-C₆)alkyloxy,
an azetidinyl optionally substituted by a (C₁-C₆)alkyloxy;
a pyrrolidin-2-one;
a 6-oxa-3-azabicyclo[3.1.1]heptane, or a 8 oxa-3-azabicyclo[3.2.1]octane;
a (C₁-C₆)alkyloxy, optionally substituted by at least one halogen, or one (C₁-C₆)alkyloxy; a halogen;
a hydroxy;
a —CN;
a —SO₂—CH₃;
a 1,1-dioxo-1,2-thiazolidin;
a cyclobutyloxy, or a tetrahydropyranyloxy;
a (C₁-C₆)alkyl optionally substituted by at least one halogen; and
a (C₁-C₆)alkyl substituted by a morpholinyl optionally substituted by at least one methyl, a 6-oxa-3-azabicyclo[3.1.1]heptane, a 8 oxa-3-azabicyclo[3.2.1]octane or a tetrahydropyranyl.

30. The method according to claim 20, wherein R₂ represents
a hydrogen;
a halogen; or
an optionally substituted (C₃-C₆)cycloalkyl.

31. The method according to claim 20, wherein said compound is selected from the group consisting of compounds of the table A.

32. The method according to claim 20, wherein said compound is selected from the group consisting of compounds of the table B.

33. The method according to claim 20, wherein R₁ is 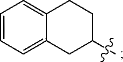
R
₂ is a hydrogen or a halogen; R₃ is a phenyl optionally substituted by a halogen; and R₅ is a hydrogen.

34. The method according to claim 20, wherein said compound is selected from the group consisting of compound #16, compound #17, compound #19, compound #57, compound #86, compound #94, compound #124, compound #140, compound #151, compound #152, compound #157, compound #162, compound #171, compound #174, compound #175, compound #180, compound #194, compound #195, compound #196, compound #B9, compound #B18 and compound #B111.

35. The method according to claim 20, wherein said compound is selected from the group consisting of compound #16, compound #17, compound #157, compound #195, and compound #196.

36. The method according to claim 20, wherein the subject has a renal fibrosis and suffers from a disease selected from the group consisting of Non-alcoholic fatty liver disease (NAFLD) and non-alcoholic steatohepatitis (NASH) and the compound has the structure of formula I, wherein R₁ is 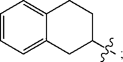
R
₂ is a hydrogen or a halogen; R₃ is a phenyl optionally substituted by a halogen; and R₅ is a hydrogen.

37. The method according to claim 20, wherein the subject has a renal fibrosis and suffers from a disease selected from the group consisting of Non-alcoholic fatty liver disease (NAFLD) and non-alcoholic steatohepatitis (NASH) and the compound is selected from the group consisting of compound #16, compound #17, compound #157, compound #195, and compound #196.

38. The method according to claim 20, wherein the subject has a renal fibrosis and suffers from a disease selected from the group consisting of Non-alcoholic fatty liver disease (NAFLD) and non-alcoholic steatohepatitis (NASH) and the compound is selected from the group consisting of compound #16 and compound #157.
